(19) United States
(12) Patent Application Publication
(10) Pub. No.: US 2018/0207380 A1
(43) Pub. Date: Jul. 26, 2018

(54) TOUCH-SENSITIVE USER INTERFACE INTENDED FOR A TRACHEOBRONCHIAL-AIR STIMULATION DEVICE

(71) Applicants: Jean-Sebastien LANTZ, Aix en Provence (FR); Adrien MITHALAL, Montpellier (FR)

(72) Inventors: Jean-Sebastien LANTZ, Aix en Provence (FR); Adrien MITHALAL, Montpellier (FR)

(21) Appl. No.: 15/743,441

(22) PCT Filed: Jul. 11, 2016
(86) PCT No.: PCT/EP2016/066454

§ 371 Date: Jan. 10, 2018

(30) Foreign Application Priority Data
Jul. 10, 2015 (FR) 1556619

(51) Int. Cl. A61M 16/00; A61B 5/091; A61B 5/00; G16H 40/60

(52) U.S. Cl. CPC A61M 16/024; A61B 5/091; A61B 5/743; A61M 16/0006; A61B 5/4839; A61M 2205/3592; A61M 2016/0027; A61M 2205/505; A61M 2205/52; A61M 2205/3553; A61M 2205/3569; G16H 40/60

(57) ABSTRACT

Disclosed is a touch-sensitive user interface intended to interact with a tracheobronchial-air stimulation device including: —a touch-sensitive display screen for providing the user with information about the use of the tracheobronchial-air stimulation device, —a communication module for communicating data between the touch-sensitive user interface and the tracheobronchial-air stimulation device, —a memory in which biofeedback software and data, referred to as input data, may be saved, and —a data processing unit configured by the biofeedback software.

### FIELD OF INVENTION

The present invention pertains to the field of the treatment of obstructive ventilatory disorders. In particular, the invention relates to a tactile user interface for a mucus stimulating device for enhancing expectoration.

### BACKGROUND OF INVENTION

There are various pathologies resulting in an obstructive ventilatory disorder, or pulmonary obstructive syndrome, which is characterized by a build-up of mucus and results in a limitation of flow rates in the respiratory tract and by an increase in air resistance.

For all these obstructive respiratory disorders, the patient has an accumulation of bronchial mucus which, because of its stagnation, is responsible for infections that can lead to serious pulmonary complications. Also, is it important to perform a regular uncluttering in these patients.

This treatment is in fact a “bronchial toilet”, the purpose of which is to avoid the superinfection that is most often the subject of these patients.

However, this bronchial toilet can be traumatic for the patient and remains of limited effectiveness when the mucus is too viscous or elastic.

Also, and in order to assist patients in this bronchial toilet, in the state of the art, various devices to facilitate the expulsion of mucus have been developed.

Some devices are known in the art, disclosing a user interface for displaying or selecting the configuration parameters of the device.

For example, this is described in the United States patent application 2009/126734, teaching a device for stimulating the tracheobronchial air of a patient. A graphical interface makes it possible to display the operational parameters and to communicate with the controller that manages the device and stores one or more therapeutic protocols.

Also, United States Patent Application 2012/285460 teaches a Mechanical Inspiration Expiration (MIE) apparatus having a fan, a steering valve, an oscillator, a mask pipe connector and a touch interface. The latter allows, through software, to control the processing cycles by means of configurable parameters in three modes: the manual m United States ode, which allows the user to decide when and how the cycles must be executed, an automatic mode, in which the device itself initiates the cycles, and a configuration mode, which makes it possible to set the amplitude of the depressions and the frequency of oscillation as well as to lock these parameters.

However, these solutions do not allow the patient to follow or influence at a real time the evolution of his treatment, that is to say during a treatment session, or over time, that is to say during several sessions. In addition, these devices do not allow the communication of medical data generated by the device to a platform dedicated to health professionals. These disadvantages can be significant with regard to the effectiveness of the treatment. Indeed, if the health professional is not free to change the treatment, it may, in the long run, lose effectiveness.

### SUMMARY

This invention thus relates to a tactile user interface intended to cooperate with a device for stimulating tracheobronchial air, characterized in that it comprises:
at least one tactile display screen making it possible to provide the user with information relating to the use of said device for stimulating tracheobronchial air;
at least one communication module enabling the communication of data between said tactile user interface and said device for stimulating tracheobronchial air;
at least one memory in which a biofeedback software and data, called input data, could be saved comprising at least:
   representative data of the depressions provided by at least one pressure sensor of said tracheobronchial air stimulation device;
   temporal data representing an expiratory time of the patient, measured by the support time, by the patient, on at least one button of at least one control or remote control of the tracheobronchial air stimulation device;
at least one data processing unit configured by the biofeedback software for:
   producing data, called scores, representative of the expiratory state of the patient at an expiration E1, based on expiratory time;
   calculating an average score per expiration session based on the scores achieved during at least one expiration during at least one session;
   transmitting the said scores and average scores according to the sessions and/or expirations on the tactile display screen in order to communicate expiratory progression to at least one patient;
   transmitting instructions for use to the patient through an analysis of acquired parameters;
   providing an interpretation of the recorded information to guide the patient in his or her proper treatment.

According to another embodiment, the communication module is a bluetooth communication module.

According to another embodiment, the tactile display screen allows, by means of the biofeedback software executed on the data processing unit and by transmission and reception of the appropriate commands, to display a screen, called “Connection” screen containing:
a virtual button “Create an account” leading after activation the program on a module of the software sending the appropriate commands to the display of a screen “creation account” allowing the user by interaction with the tactile user interface to display on the tactile display screen the information needed to create a user account and then save the information of the user account to the memory;
a virtual button “Change account” leading after activation the program on a module of the software sending the appropriate commands to the display of a “change user” screen allowing the user by interaction with the tactile user interface to display on the tactile display screen the information needed to change the user account and then save the change information to the memory;
a virtual button “Go” leading after activating the program on a module of the software “Progress” sending the appropriate commands to display a “Progress” screen allowing the user to follow his progress over sessions.

According to another embodiment, the “Create an account” screen contains the following elements:
“identity” information that the user must provide by means of fields that can be filled;
“session” information that the user selects through drop-down menus;
a field that can be filled “Max pressure” associated with a virtual button “Take the test” leading after activation the program on a module of the software sending the appropriate commands to the execution of a pressure test allowing the user, by interacting with the tactile user interface, to test different pressures and then record on the memory said selected pressure at the end of the test;
a virtual button “Save” leading after activation the program on a module of the software sending the appropriate commands to the registration of said user account in said memory allowing the user by interaction with the tactile user interface to register an account;
a virtual button “Delete” leading after activation the program on a module of the software sending the appropriate commands to the deletion of the information entered by the user allowing the user by interaction with the tactile user interface to delete the information entered by the user. on the “Create an Account” screen.
   According to another embodiment, the “identity” information may be surname, first name, age and sex and “session” information may be a difficulty and a number of breaths per session.

According to another embodiment, the “Progress” screen contains the following elements:
a user identity;
a virtual button “Create an account” leading after activation the program on a module of the software sending the appropriate commands to the display of a “creation account” screen allowing the user by interaction with the tactile user interface to display on the tactile display screen the information needed to create a user account and then save the information of the user account to the memory;
a virtual button “Change account” leading after activation the program on a module of the software sending the appropriate commands to the display of a “change user” screen allowing the user by interaction with the tactile user interface to display on the tactile display screen the information needed to change the user account and then save the change information to the memory;
a virtual button “Parameters” leading after activation the program on a module of the software sending the appropriate commands to the setting allowing the user by interaction with the tactile user interface to adjust the parameters of said tactile user interface;
a progression curve with the average score on the ordinates and the number of sessions on the abscissa;
a virtual button “New session” leading after activation the program on a module of the software sending the appropriate commands to the display of a “session” screen allowing the user by interaction with the tactile user interface to display on the tactile display screen information necessary for the conduct of a session and then save on the memory information session.

According to another embodiment, the average score is calculated by the data processing unit by performing an output of a mean pressure and an average time recorded during at least one session.

According to another particularity, the “session” screen contains the following elements:
a virtual pressure gauge representing the evolution of the data representative of the pressures contained in the input data;
a virtual chronometer representing the evolution of the temporal data contained in the input data;
a progression curve representing scores on the ordinates and expirations on the abscissa;
an average score calculated over the course of the session;
informative indications concerning the course of the session;
a virtual button “cancel” leading after activation the program on a module of the software sending the appropriate commands to cancel a session allowing the user by interaction with the tactile user interface to end the session.

According to another embodiment, the “session” screen also contains “Pop-up” type indications, produced by the data processing unit and transmitted to the tactile display screen by means of the biofeedback software, for guiding the patient during his session.

According to another embodiment, all the data processed on the tactile user interface can be transmitted, by means of the communication module, to a secure platform.

According to another feature, the tactile user interface provides a didactic visual to the patient at the beginning of his first session.

### BRIEF DESCRIPTION OF THE DRAWINGS

FIG. 1 represents a diagram of the operation of the interface.

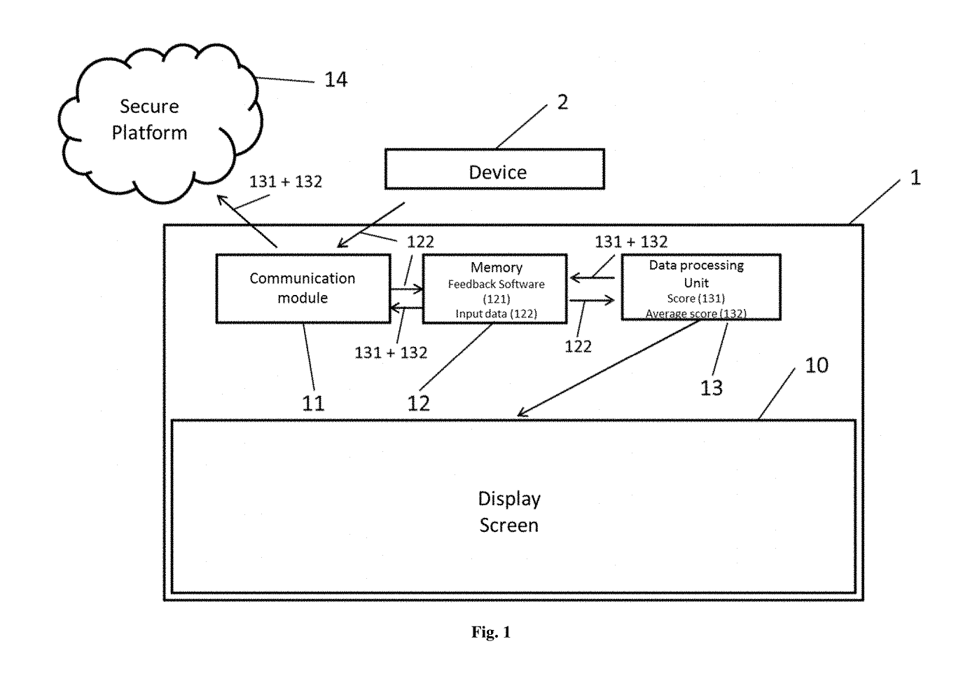

FIG. 2 represents an example of a connection screen.

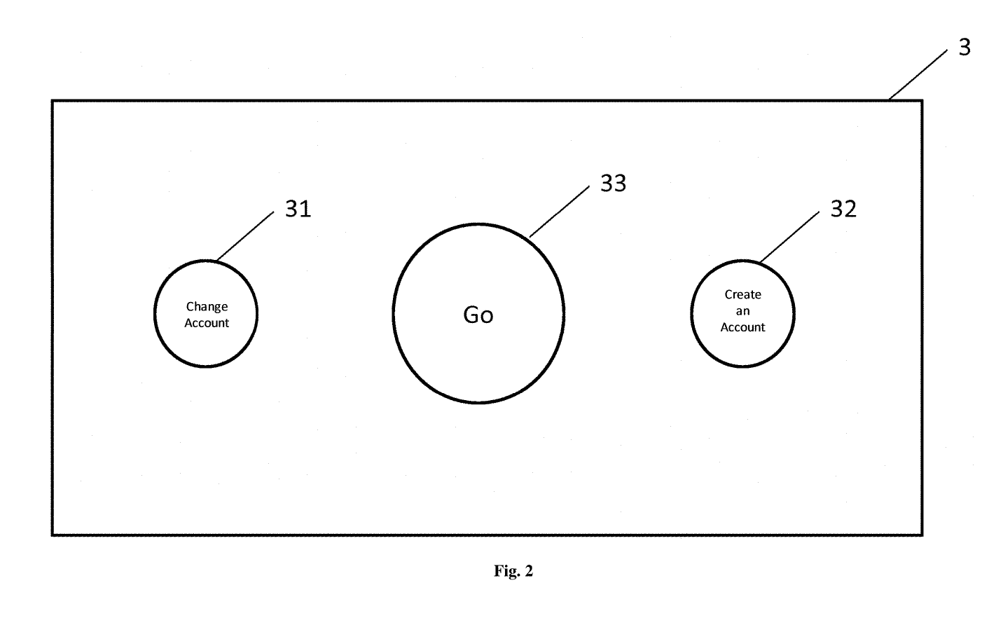

FIG. 3 represents an example of an account creation screen.

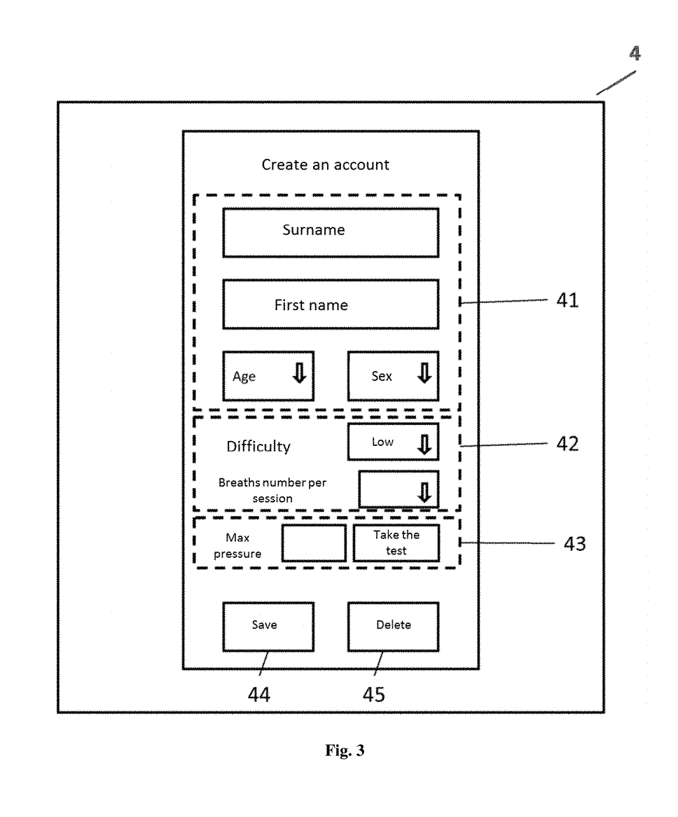

FIG. 4 represents an example of a progression screen.

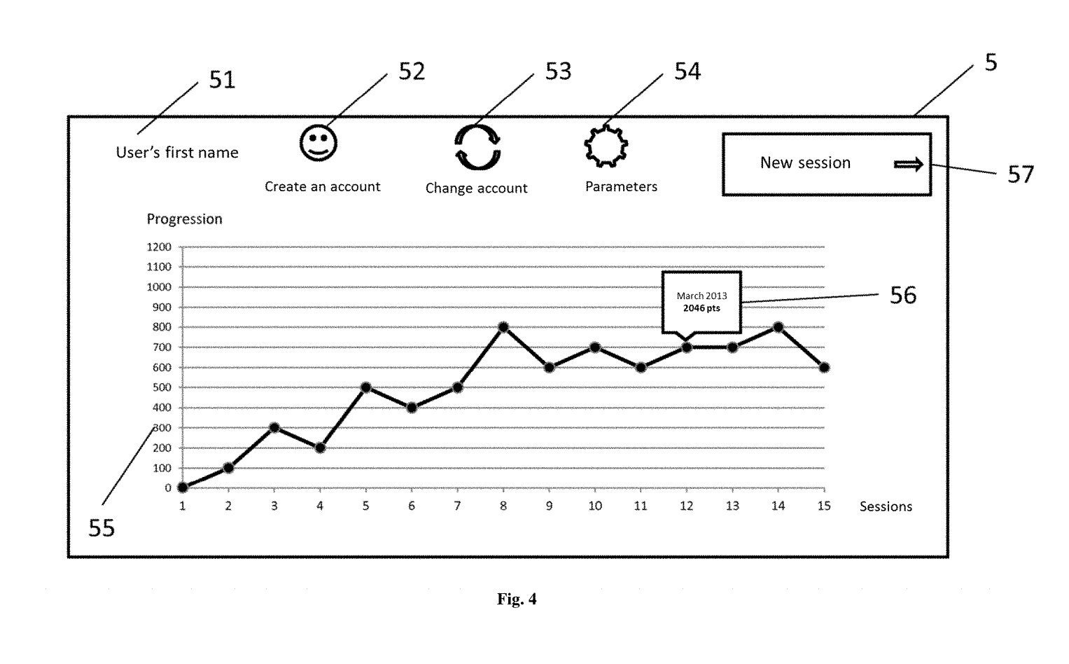

FIG. 5 represents an example of a session screen.

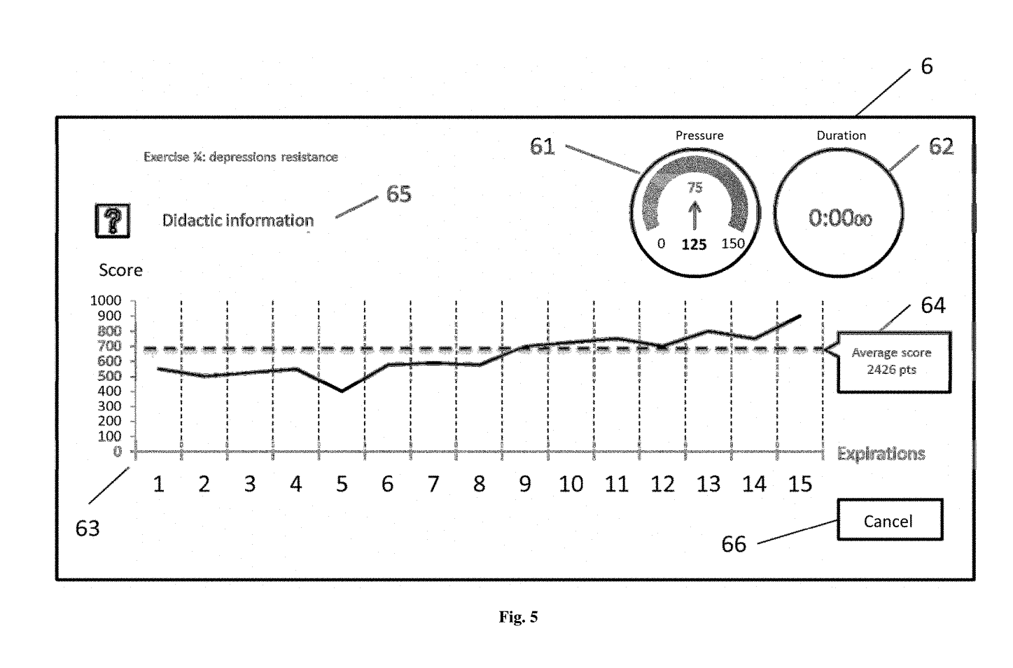

### DETAILED DESCRIPTION

This invention relates to a tactile user interface (1) for cooperating with a device for stimulating tracheobronchial air (2).

In some embodiments, the tactile user interface (1) has at least one tactile display screen (10) for providing the user with information relating to the use of said tracheobronchial air stimulation device (2). Preferably, said tactile display screen (10) is large enough, of the order of 9 inches, to provide the user with a simple and effective use of the interface.

In some embodiments, said tactile user interface (1) includes a communication module (11) for communicating data between said tactile user interface (1) and said tracheobronchial air stimulating device (2). Preferably, said communication module (11) is in bluetooth connection with a communication module (11) integrated in the device for stimulating tracheobronchial air (2). This bluetooth link enables the tactile user interface (1) to recover data generated by the tracheobronchial air stimulation device (2), in particular for example pressure measurements carried out by a pressure sensor or else temporal data related to the stopwatch integrated in the device for stimulating tracheobronchial air (2).

In some embodiments, said tactile user interface (1) includes a memory (12). This contains a preinstalled biofeedback software (121) as well as data called input data (122) comprising data representative of pressures provided by at least one pressure sensor of said tracheobronchial air stimulation device (2). These data preferably represent the intra-pulmonary depression of the patient and are measured by means of a pressure sensor integrated on the device. The recovery of these data of intra-pulmonary depression makes it possible in particular to follow the evolution of the treatment of the patient. Indeed, if the intra-pulmonary depression increases well over the expiration, this indicates that the treatment is carried out effectively. The input data (122) also includes time data, which preferably represents the expiratory time of the patient. This expiratory time preferably corresponds to the duration of pressure on the start button of a remote control of the tracheobronchial air stimulation device (2). Indeed, it is proposed to measure the expiration time by involving the patient who himself presses the button to indicate the expiration whose duration is thus measured. This measure has the advantage of dispensing with a flow meter which is generally not reliable enough for this type of values of depressions and frequencies. This measurement is therefore based on an adjustment of the suction flow rate of the machine and a measurement of the suction time (and therefore of expiration by the patient), called expiratory time, by the patient himself, who is asked to exhale more than it inspires. Thus, as expiratory time, the lungs are emptied and depression becomes more important because of the decrease in lung volume. The measurement of the volume of air drawn by the machine (expiratory volume) and its comparison with the inspiratory volume makes it possible to set objectives for the patients, in particular to go down in the low lung volumes. The work in these low volumes allows a strong mobilization of mucus in the distal airways. Indeed, it has been observed that if this mucus stagnates, it becomes responsible for pulmonary infections, the leading cause of death for patients with obstructive pathologies. The interaction of the patient with the user interface (1) thus allows the patient to be involved in measuring and achieving objectives, which improves the efficiency and/or the reliability of the treatment. In addition, the measurement of exhalation time is relevant in the monitoring of the patient's condition, in particular because a fall of this time can mean decompensations of the patients.

In some embodiments, said tactile user interface (1) includes a data processing unit (13) configured by the biofeedback software (121). This data processing unit (13) is the link between the memory (12) and the tactile display screen (10). This calculates the different data to be represented on the tactile display screen (10) by means of the biofeedback software (121) to produce preferably several screens. In particular, the so-called scores (131) are calculated according to the following formula: Depression×Time per expiration.

For example, for an E1 expiration of duration 3 seconds and a depression of 125 millibars, one obtains a score of 375 points.

Thus, the data processing unit (13) can calculate an average score (132) per expiration session based on the scores achieved during at least one expiration during at least one session. This average score (132) is calculated according to the following formula: Average Depression×Average Time.

Finally, the treatment unit (13) transmits said scores (131) and average scores (132) according to the sessions and or expirations on the tactile display screen (10) to communicate its expiratory progression to the least one patient. The said treatment unit also transmits instructions for use to the patient through an analysis of acquired parameters and provides an interpretation of the recorded information to guide the patient in his or her proper treatment.

It is thus clear that the present application relates in fact to a tactile user interface (1), ie a device or system comprising computer means including data processing unit (13), means for storage (12) and physical interaction means (10) (or human-machine interface) with the user (preferably a tactile display screen, even if any combination of display means and input means is possible). In addition, this type of user interface may not be tactile and be controlled by the voice or eyes without departing from the scope of the invention, even if the entries or selections of the user would not then be made by a “pressing” in the strict sense.

In addition, the invention relates to this interface (1) as such, whether or not integrates the device for stimulating tracheobronchial air (2) to which it is intended to be connected. Indeed, the interface (1) is preferably connectable to a device via a wireless link but a wired connection is possible and the present application does not detail those aspects that are within the reach of the skilled person. Thus, this interface (1) is defined as being connectable to this type of stimulation device (2) because it is during their combined use of these two devices that the invention can be effectively implemented, even if most all or some of the specific characteristics are rather implemented in the interface (1) than in the stimulation device (2). In particular, reference is made in the present application to supports or selections, especially for the measurement of expiratory time and it should be clear to the skilled person that this measure through the pressing of a button on a remote control because it is generally preferred to use a device that is easily grippable and usable by the patient, such as a remote control or a simple push button for example. However, this action on the remote control can be replaced or complemented by pressing a virtual button or any other physical action captured by any type of sensor providing the data processing unit (13) the data relating to the support time. It is of course the same for the other data generated following the actions of the user, whether the patient or qualified personnel preparing or configuring the interface (1) and/or the device (2) of stimulation.

In some embodiments, the tactile display screen (10) allows, by means of the biofeedback software (121) executed on the data processing unit (13) and by transmission and reception of the appropriate commands, to display a screen, called “Connection” screen (3) containing:
a virtual button “Create an account” (32) leading after activation the program on a module of the software sending the appropriate commands to the display of a “creation account” screen (4) allowing the user by interaction with the tactile user interface (1) displaying on the tactile display screen (10) the information necessary for the creation of a user account and then saving the user account information on the memory (12);
a virtual button “Change account” (31) leading after activation the program on a module of the software sending the appropriate commands to the display of a “change user” screen allowing the user by interaction with the tactile user interface (1) displaying on the tactile display screen (10) the information necessary to change the user account and then save on the memory (12) the change information;
A virtual button “Go” (33) leading after activation the program on a module of the software “Progress” sending the appropriate commands to display a “Progress” screen (5) allowing the user to follow his progress during the sessions.

Preferably, the virtual button “Go” (33) is more imposing than the virtual buttons “Create an account” (32) and “Change account” (31) to guide the user who already has an account registered on said interface tactile user interface (1) faster to his “Progress” screen (5).

In some embodiments, the “Create Account” screen (4) contains the following elements:
“identity” information (41) that the user must provide by means of fields that can be filled;
session information (42) that the user selects through drop-down menus;
a field that can be filled “Max pressure” associated with a virtual button “Test” (43) leading after activation the program on a module of the software sending the appropriate commands to the execution of a pressure test allowing the user by interaction with the tactile user interface (1) to test different pressures and then record on the memory (12) said selected pressure at the end of the test;
a virtual button “Save” (44) plugging after activation the program on a module of the software sending the appropriate commands to the registration of said user account in said memory (12) allowing the user by interaction with the tactile user interface (1) to register an account;
a virtual button “Delete” (45) leading after activation the program on a module of the software sending the appropriate commands to the deletion of the information entered by the user allowing the user by interaction with the tactile user interface (1) of delete the information entered by the user on said “Create account” screen (4).

“Identity” information (41) can be surname, first name, age and sex and “session” information (42) can be a difficulty and a number of breaths per session. Preferably, age, sex, difficulty, and number of breaths per session are in the form of a drop-down menu.

In some embodiments, the “Progress” screen (5) contains the following elements:
a user identity (51);
a virtual button “Create an account” (52) leading after activation the program on a module of the software sending the appropriate commands to the display of a “creation account” screen (4) allowing the user by interaction with the tactile user interface (1) to display on the tactile display screen (10) the information necessary for the creation of a user account and then to save the information of the user account on the memory (12);
a virtual button “Change account” (53) leading after activation the program on a module of the software sending the appropriate commands to the display of a “change user” screen allowing the user by interaction with the tactile user interface (1) displaying on the tactile display screen (10) the information necessary to change the user account and then save on the memory (12) the change information;
a virtual button “Parameters” (54) leading after activation the program on a module of the software sending the appropriate commands to the setting allowing the user by interaction with the tactile user interface (1) to adjust the parameters of said touch tactile user interface (1);
a progression curve (55) with the average score (132) on the ordinate and the number of sessions on the abscissa;
a virtual button “New session” (57) leading after activation the program on a module of the software sending the appropriate commands to the display of a “session” screen (6) allowing the user by interaction with the tactile user interface (1) to display on the tactile display screen (10) the information necessary for the conduct of a session and then save on the memory (12) the information of the session.

Preferably, the progression curve is plotted in connected points, which allows the user to view the average score (56) achieved during a given session and the date of said session by pressing his finger on the tactile user interface (1) at said point.

In some embodiments, the “session” screen (6) contains the following elements:
a virtual pressure gauge (61) representing the evolution of the data representative of the pressures contained in the input data (122);
a virtual chronometer (62) representing the evolution of the temporal data contained in the input data (122);
a progression curve (63) representing scores (131) on the ordinate and expirations on the abscissa;
an average score (64) calculated over the course of the session;
informative indications (65) relating to the conduct of the session;
a virtual button “cancel” (66) leading after activation the program on a module of the software sending the appropriate commands to the cancellation of a session allowing the user by interaction with the tactile user interface (1) to put a term at the said meeting.

At the launch of a session, commands are sent to allow the display of the “session” screen (6), the retrieval of information necessary for the representation of a virtual gauge and the materialization on this virtual gauge of the pressures. suffered by the patient. Preferably, the virtual pressure gauge (61) averages a number of samples measured by a pressure sensor of the tracheobronchial air stimulation device (2) sampled at 100 Hz.

These commands also make it possible to retrieve the information necessary for the representation of a virtual chronometer and the materialization on this virtual chronometer of the expiratory time of the patient.

The average score (64) is displayed as a dotted line on the progression curve.

In some embodiments, the “session” screen (6) also contains “Pop-up” type indications produced by the data processing unit (13) and transmitted to the tactile display screen (10) by means of biofeedback software (121), to guide the patient during his session. For example, if the expiratory time displayed by the stopwatch is less than 6 seconds, a Pop-up indication proposes to lower the amplitude of the depressions to increase the expiratory time. Similarly, if the expiratory time displayed by the stopwatch is greater than 9 seconds, a Pop-up indication proposes to increase the amplitude of the depressions to improve the effectiveness of the treatment.

In some embodiments, all the data processed on the tactile user interface (1) can be transmitted, by means of the communication module (11), to a secure platform (14). Thus, the health professional has, through the secure platform (14), all of its clientele, alerts indicating patients in regression and a means of communication with said patients.

In some embodiments, the tactile user interface (1) provides a didactic visual to the patient at the beginning of his or her first session. Said instructional visual introduces the patient a user guide of the tactile user interface (1) to facilitate the progress of its treatment.

The present application describes various technical features and advantages with reference to the figures and/or various embodiments. Those skilled in the art will understand that the technical features of a given embodiment may in fact be combined with features of another embodiment unless the reverse is explicitly mentioned or it is evident that these characteristics are incompatible or that the combination does not provide a solution to at least one of the technical problems mentioned in this application. In addition, the technical features described in a given embodiment can be isolated from the other features of this mode unless the opposite is explicitly mentioned.

It should be obvious to those skilled in the art that the present invention allows embodiments in many other specific forms without departing from the scope of the invention as claimed. Therefore, the present embodiments should be considered by way of illustration, but may be modified in the field defined by the scope of the appended claims, and the invention should not be limited to the details given above.

1-11. (canceled)

12. A tactile user interface (1) for cooperating with a device for stimulating tracheobronchial air (2), comprising:
at least one tactile display screen (10) making it possible to provide the user with information relating to the use of said device for stimulating tracheobronchial air (2);
at least one communication module (11) enabling the communication of data between said tactile user interface (1) and said device for stimulating tracheobronchial air (2);
at least one memory (12) in which a biofeedback software (121) and data, called input data (122), could be saved comprising at least:
representative data of the depressions provided by at least one pressure sensor of said tracheobronchial air stimulation device (2);
temporal data representing an expiratory time of the patient, obtained by the support time measurement, by the patient, on at least one button of at least remote control of the tracheobronchial air stimulation device (2);
at least one data processing unit (13) configured by the biofeedback software (121) for:
producing data, called scores (131), representative of the expiratory state of the patient at an expiration E1, based on the expiratory time;
calculating an average score (132) per expiry session based on the scores achieved during at least one expiration during at least one session;
transmitting the said scores (131) and average scores (132) according to the sessions and/or expirations on the tactile display screen (10) in order to communicate expiratory progression to at least one patient;
transmitting instructions for use to the patient through an analysis of acquired parameters;
providing an interpretation of the recorded information to guide the patient in his or her proper treatment.

13. The tactile user interface (1) according to claim 12, wherein the communication module is a bluetooth communication module.

14. The tactile user interface (1) according to claim 12, wherein the tactile display screen (10) allows, by means of the biofeedback software (121) executed on the data processing unit (13) and by transmission and reception of the appropriate commands, to display a screen, called “Connection” screen (3) containing:
a virtual button “Create an account” (32) leading after activation the program on a module of the software sending the appropriate commands to the display of a “creation account” screen (4) allowing the user by interaction with the tactile user interface (1) to display on the tactile display screen (10) the information needed to create a user account and then save the information of the user account to the memory (12);
a virtual button “Change account” (31) leading after activation the program on a module of the software sending the appropriate commands to the display of a “change user” screen allowing the user by interaction with the tactile user interface (1) to display on the tactile display screen (10) the information needed to change the user account and then save the change information to the memory (12);
a virtual button “Go” (33) leading after activating the program on a module of the software “Progress” sending the appropriate commands to display a “Progress” screen (5) allowing the user to follow his progress over sessions.

15. The tactile user interface (1) according to claim 14, wherein “Create an account” screen (4) contains the following elements:
“identity” information (41) that the user must provide by means of fields that can be filled;
“session” information (42) that the user selects through drop-down menus;
a field that can be filled “Max pressure” associated with a virtual button “Take the test” (43) leading after activation the program on a module of the software sending the appropriate commands to the execution of a pressure test allowing the user, by interacting with the tactile user interface (1), to test different pressures and then record on the memory (12) said selected pressure at the end of the test;
a virtual button “Save” (44) leading after activation the program on a module of the software sending the appropriate commands to the registration of said user account in said memory (12) allowing the user by interaction with the tactile user interface (1) to register an account;
a virtual button “Delete” (45) leading after activation the program on a module of the software sending the appropriate commands to the deletion of the information entered by the user allowing the user by interaction with the tactile user interface (1) to delete the information entered by the user. on the “Create an Account” screen (4).

16. The tactile user interface (1) according to claim 15, wherein the “identity” information (41) may be surname, first name, age and sex and “session” (42) information may be a difficulty and a number of breaths per session.

17. The tactile user interface (1) according to claim 14, wherein the “Progress” screen (5) contains the following elements:
a user identity (51);
a virtual button “Create an account” (52) leading after activation the program on a module of the software sending the appropriate commands to the display of a “creation account” screen (4) allowing the user by interaction with the tactile user interface (1) to display on the tactile display screen (10) the information needed to create a user account and then save the information of the user account to the memory (12);
a virtual button “Change account” (53) leading after activation the program on a module of the software sending the appropriate commands to the display of a “change user” screen allowing the user by interaction with the tactile user interface (1) to display on the tactile display screen (10) the information needed to change the user account and then save the change information to the memory (12);
a virtual button “Parameters” (54) leading after activation the program on a module of the software sending the appropriate commands to the setting allowing the user by interaction with the tactile user interface (1) to adjust the parameters of said tactile user interface (1);
a progression curve (55) with the average score (132) on the ordinates and the number of sessions on the on the abscissa;
a virtual button “New session” (57) leading after activation the program on a module of the software sending the appropriate commands to the display of a “session” screen (6) allowing the user by interaction with the tactile user interface (1) to display on the tactile display screen (10) information necessary for the conduct of a session and then save on the memory (12) information session.

18. The tactile user interface (1) according to claim 12, wherein the average score (132) is calculated by the data processing unit (13) by performing an output of a mean pressure and an average time recorded during at least one session.

19. The tactile user interface (1) according to claim 17, wherein the “session” screen (6) contains the following elements:
a virtual pressure gauge (61) representing the evolution of the data representative of the pressures contained in the input data (122);
a virtual chronometer (62) representing the evolution of the temporal data contained in the input data (122);
a progression curve (63) representing scores (131) on the ordinates and expirations on the abscissa;
an average score (64) calculated over the course of the session;
informative indications (65) concerning the course of the session;
a virtual button “cancel” (66) leading after activation the program on a module of the software sending the appropriate commands to cancel a session allowing the user by interaction with the tactile user interface (1) to end the session.

20. The tactile user interface (1) according to claim 17, wherein the “session” screen (6) also contains “Pop-up” type indications, produced by the data processing unit (13) and transmitted to the tactile display screen (10) by means of the biofeedback software (121), for guiding the patient during his session.

21. The tactile user interface (1) according to claim 12, wherein all the data processed on the tactile user interface (1) can be transmitted, by means of the communication module (11), to a secure platform (14).

22. The tactile user interface (1) according to claim 11, wherein the tactile user interface (1) provides a didactic visual to the patient at the beginning of his first session.

23. A tracheobronchial air stimulation device (2), further comprising at least one tactile user interface (1) according to claim 11.

24. The tactile user interface (1) according to claim 14, wherein all the data processed on the tactile user interface (1) can be transmitted, by means of the communication module (11), to a secure platform (14).

25. The tactile user interface (1) according to claim 15, wherein all the data processed on the tactile user interface (1) can be transmitted, by means of the communication module (11), to a secure platform (14).

26. The tactile user interface (1) according to claim 16, wherein all the data processed on the tactile user interface (1) can be transmitted, by means of the communication module (11), to a secure platform (14).

27. The tactile user interface (1) according to claim 17, wherein all the data processed on the tactile user interface (1) can be transmitted, by means of the communication module (11), to a secure platform (14).

28. The tactile user interface (1) according to claim 18, wherein all the data processed on the tactile user interface (1) can be transmitted, by means of the communication module (11), to a secure platform (14).

29. The tactile user interface (1) according to claim 19, wherein all the data processed on the tactile user interface (1) can be transmitted, by means of the communication module (11), to a secure platform (14).

30. The tactile user interface (1) according to claim 20, wherein all the data processed on the tactile user interface (1) can be transmitted, by means of the communication module (11), to a secure platform (14).

31. The tactile user interface (1) according to claim 12, wherein the tactile user interface (1) provides a didactic visual to the patient at the beginning of his first session.
